(19) United States
(12) Plant Patent Application Publication
(10) Pub. No.: US 2020/0275596 P1
(43) Pub. Date: Aug. 27, 2020

(54) KIWI VARIETY NAMED `RED GOAL'

(50) ;

(71) Applicant: SUMMERFRUIT S.r.l., Castel Bolognese (IT)

(72) Inventor: Giampaolo Dal Pane, Castel Bolognese (IT)

(21) Appl. No.: 16/873,231

(22) Filed: Feb. 27, 2020

(30) Foreign Application Priority Data
Feb. 27, 2019 (EU) 2019/0552

(51) Int. Cl. A01H 6/00

(52) U.S. Cl. PLT/156

(57) ABSTRACT

A new and distinct kiwi variety of Actinidia chinensis Planchon named ‘RED GOAL’, particularly characterized by distinctive size of fruit, late time of maturity for harvesting, and long cold storage life. Other desirable characteristics include fruits with red pulp and green/brown, almost hairless skin.

Latin name of the genus and species: Actinidia chinensis Planchon.

Variety denomination: ‘RED GOAL’.

### BACKGROUND OF THE INVENTION

The disclosure provides a new and distinct cultivar of kiwi varieties, botanically known as Actinidia chinensis Planchon, and hereinafter referred to by the name ‘RED GOAL’.

The new cultivar is a product of accidental discovery in Italy under the direction of Giampaolo DAL PANE, an Italian citizen. Several seeds of different and unknown origins were planted. The new variety was discovered in Italy in 2016; it is not the result of any known crossing.

Fruit of the new variety was first evaluated in year 2016 with favorable results. After the first evaluation, semi-hardwood cuttings were made of ‘RED GOAL’ and were grafted onto Hayward and Tomuri (selection 258), as rootstocks. Evaluation, asexual propagation, and grafting all first took place at the nursery in Santa Cristina e Bissone (Pavia), Italy. Subsequent evaluations of the variety have shown the characteristics to be true to type.

### SUMMARY OF THE INVENTION

The cultivar ‘RED GOAL’ has not been observed under all possible environmental conditions. The phenotype may vary somewhat with variations in environment, such as temperature, day length, and light intensity, without, however, any variance in genotype.

The following traits of ‘RED GOAL’ have been repeatedly observed and are determined to be the unique characteristics of ‘RED GOAL’. These characteristics, in combination, distinguish ‘RED GOAL’ as a new and distinct Actinidia chinensis Planchon cultivar with respect to other red pulp Actinidia:
1. Distinctive size of fruit;
2. Late time of maturity for harvesting; and
3. Long cold storage life.

### COMPARISON TO RED PULP ACTINIDIA

Plants of the new cultivar ‘RED GOAL’ are similar to plants of the seed, Actinidia chinensis Planchon Hong-Yang and Dong-Hong in most horticultural characteristics. However, plants of the new cultivar ‘RED GOAL’ produce fruit several weeks (roughly three weeks) later than Hong-Yang and Dong-Hong. Average fruit size of ‘RED GOAL’ is about 100 grams compared to other red pulp Actinidia chinensis (e.g. Hong-Yang and Dong-Hong) which average fruit weight ranges from about 75 to 80 g. The cold storage life of ‘RED GOAL’ is about 5 months, whereas for Hong-Yang and Dong-Hong is about 3 months.

### COMMERCIAL COMPARISON

‘RED GOAL’ has fruits with red pulp and green/brown almost hairless skin. On the other hand, Hayward has green pulp and very hairy brown skin.

### BRIEF DESCRIPTION OF THE DRAWING

The accompanying (drawings) illustrate the overall appearance of the new Actinidia chinensis Planchon ‘RED GOAL’ showing the colors, as true as is reasonably possible with digital reproduction. Colors in the photographs may differ slightly from the color values cited in the detailed botanical description, which accurately describe the color of ‘RED GOAL’. All photographs are taken of plants that are approximately 3 years old. The photographs were taken using conventional techniques and although colors may appear different from actual colors due to light reflectance the colors are as accurate as possible by conventional photographic techniques.

FIGS. 1 and 2 illustrate in full color typical branches and buds on plants of ‘RED GOAL’.

FIGS. 3-4 illustrate in full color typical foliage of ‘RED GOAL’.

FIGS. 5-8 illustrate in full color typical foliage, hanging fruit, and stems on plants of ‘RED GOAL’.

FIGS. 9-11 illustrate in full color typical fruit harvested from ‘RED GOAL’.

FIG. 12 illustrates in full color an enlargement of the pulp of typical fruit harvested from ‘RED GOAL’.

FIG. 13 illustrates in full color flowers of ‘RED GOAL’.

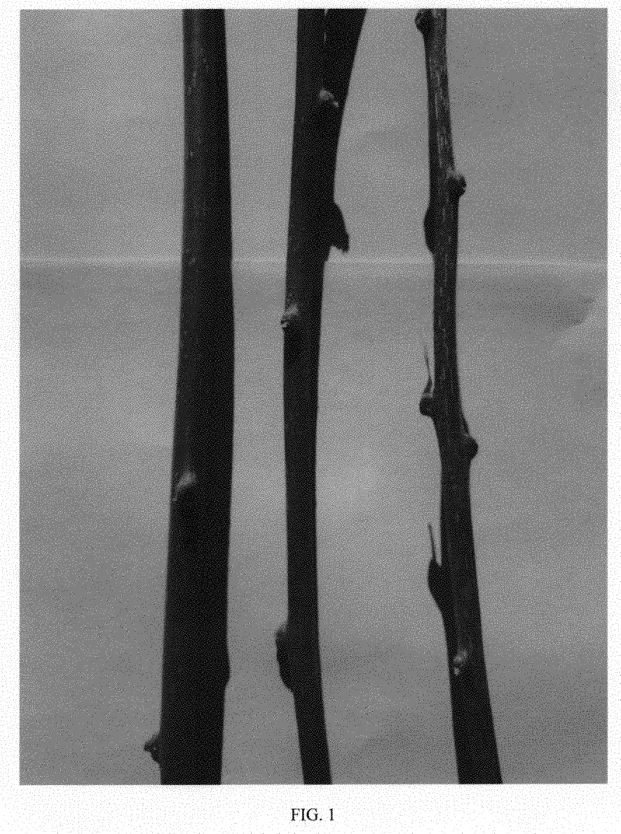

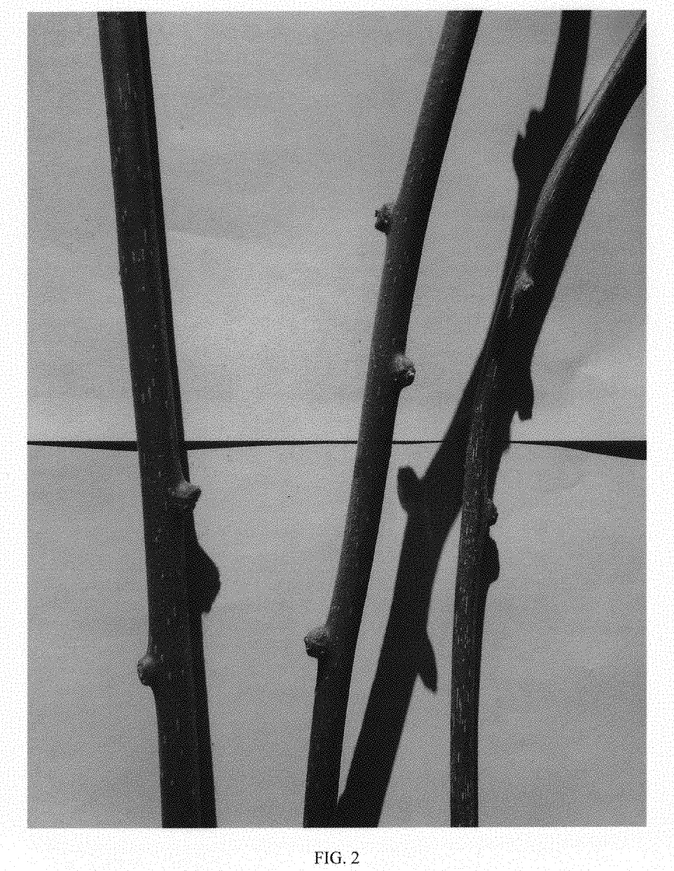

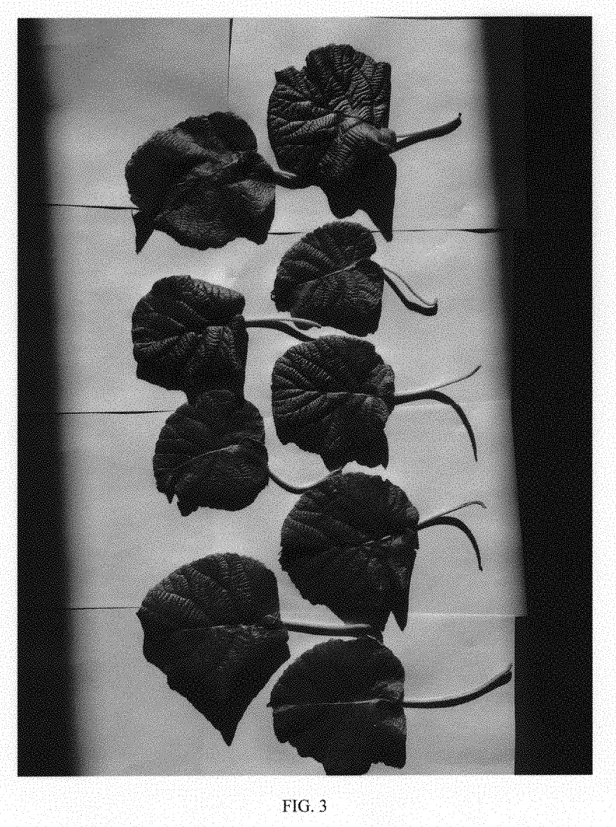

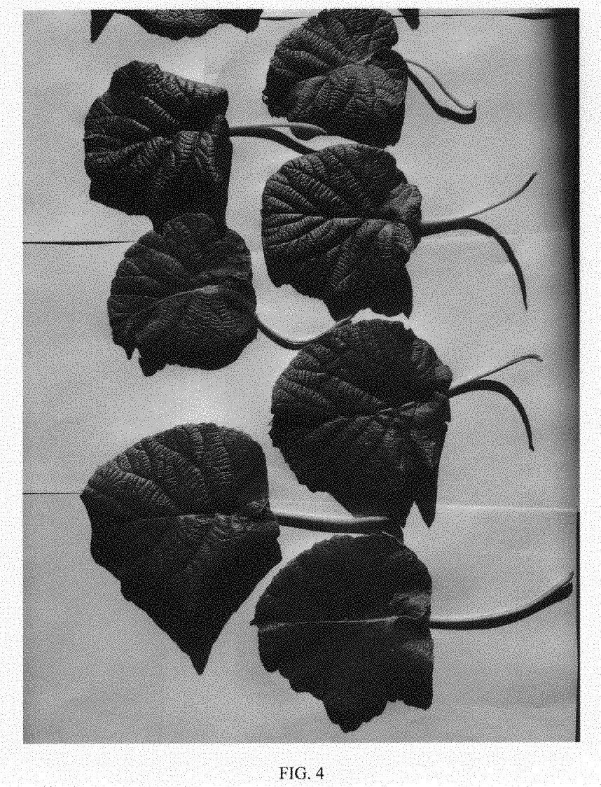

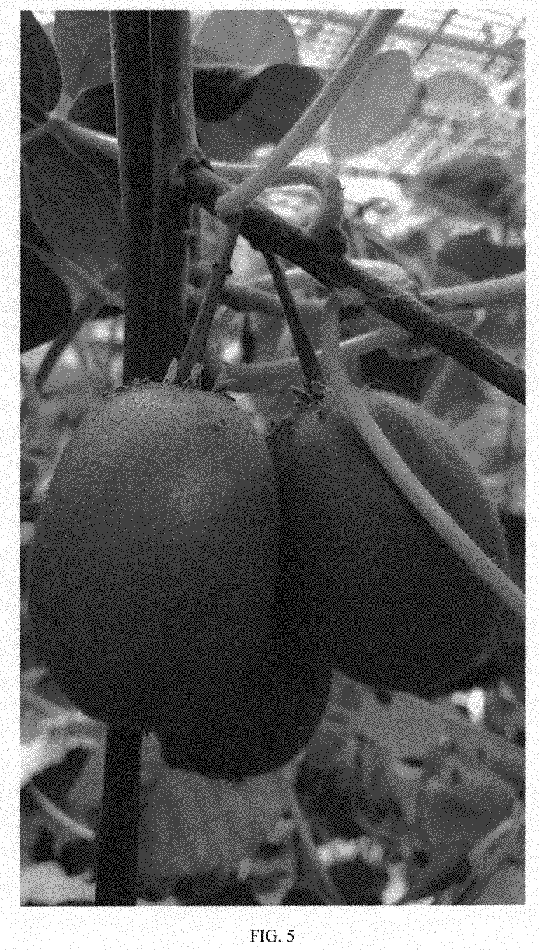

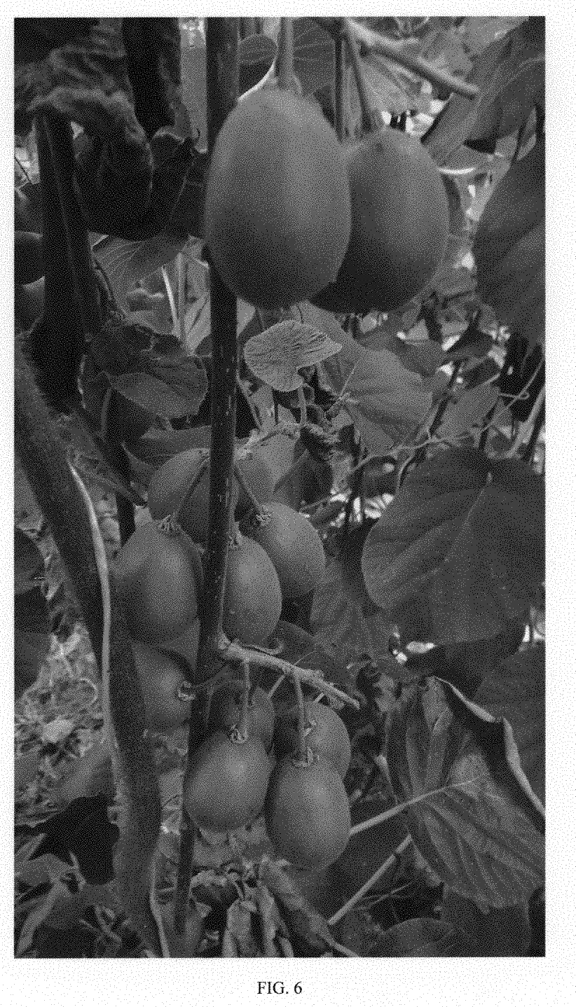

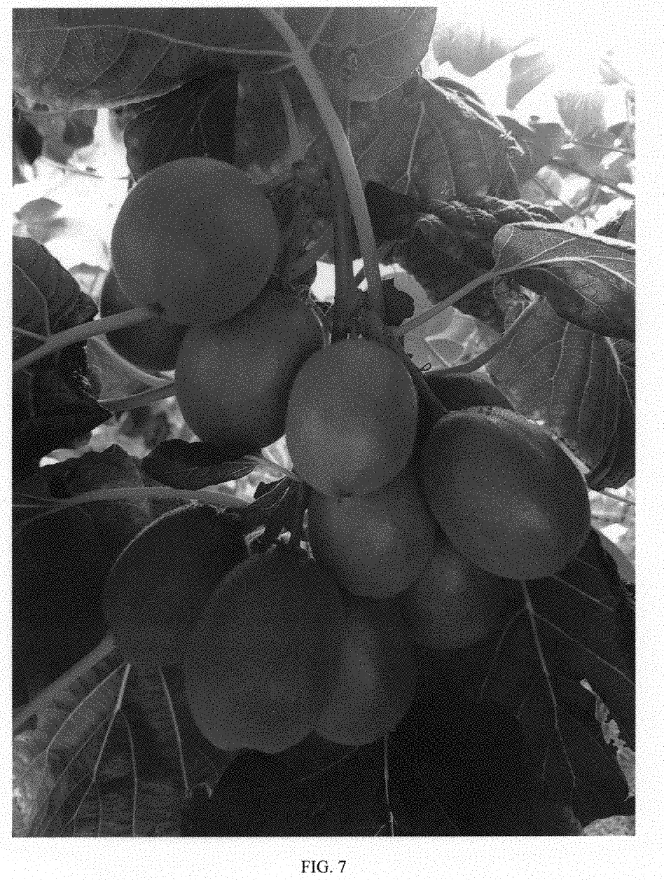

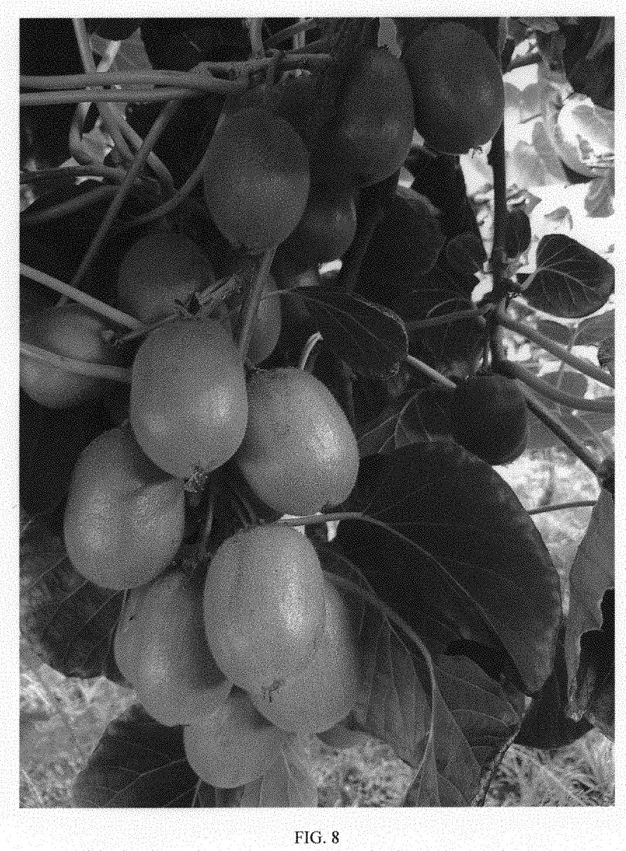

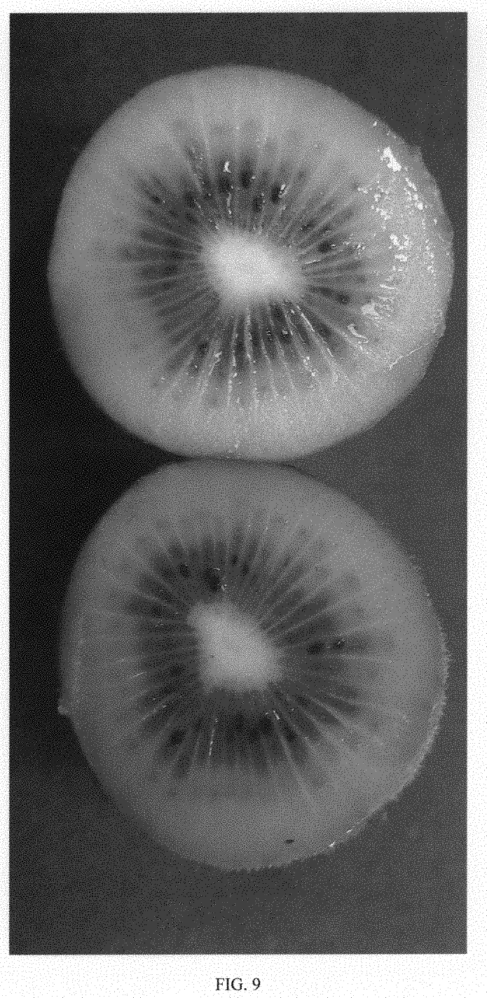

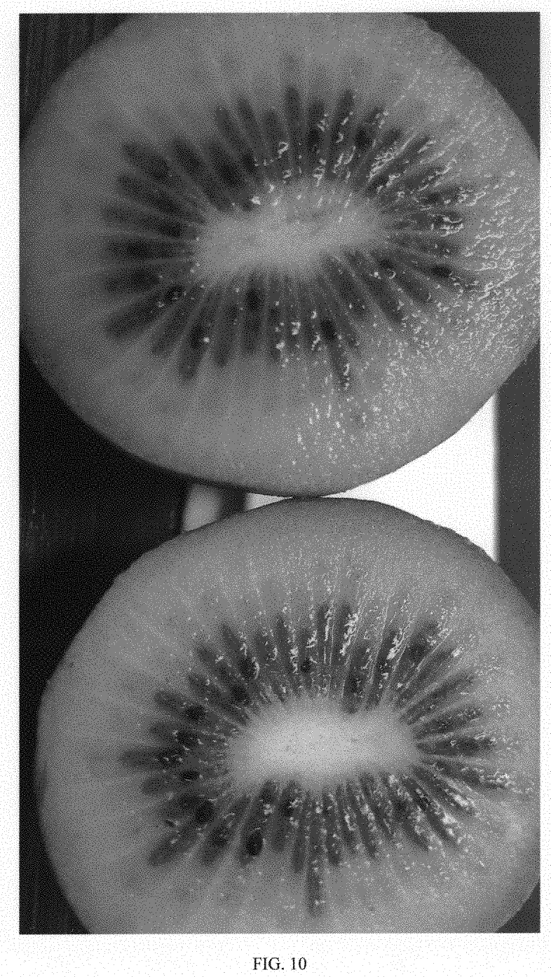

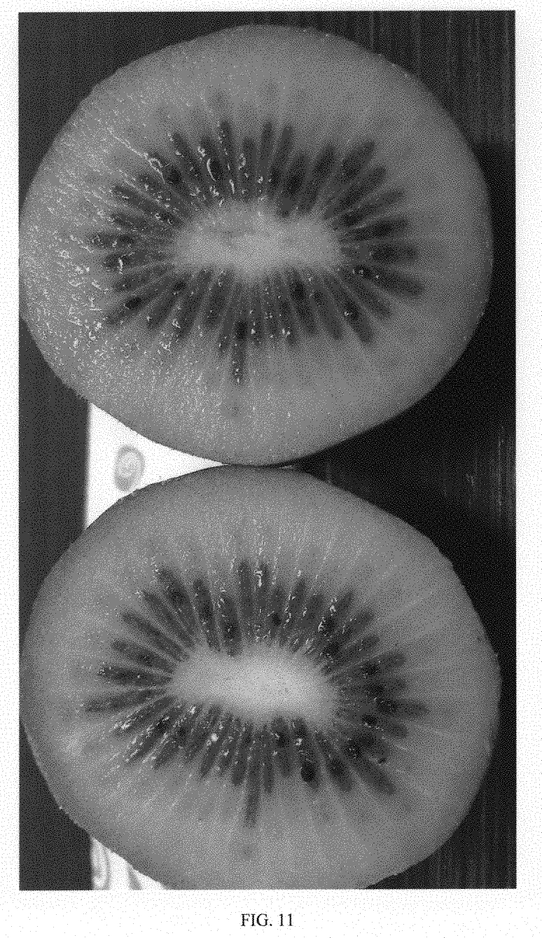

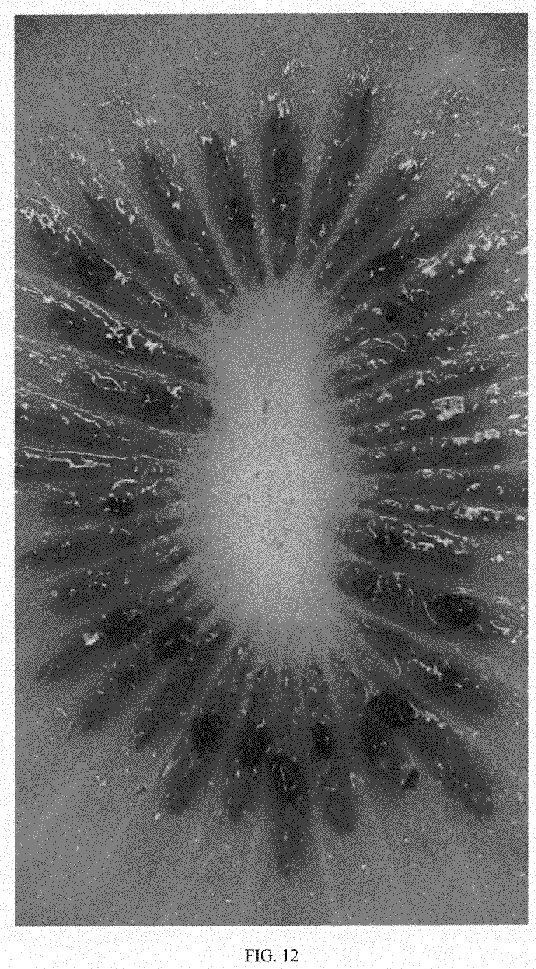

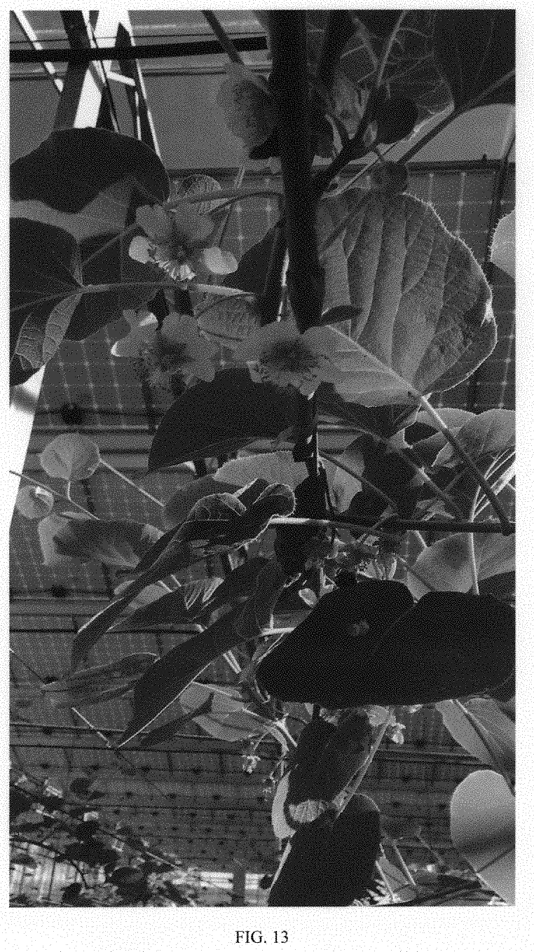

### DETAILED BOTANICAL DESCRIPTION

The following observations and measurements describe ‘RED GOAL’ plants grown in greenhouse under commercial trial conditions in Santa Cristina e Bissone (Pavia), Italy. The growing temperature ranged from a minimum of −10° C. in winter (January) up to 40-42° C. in summer (July-August). Light differences between day and night during the summer days. Annual rainfall is approximately 600 mm per year. Measurements and numerical values represent an average of 10 typical plant types. Botanical classification: Actinidia chinensis Planchon ‘RED GOAL’.
Classification:
   Botanical.—Actinidia chinensis Planchon.
Propagation: ‘RED GOAL’ can be successfully grafted onto rootstocks of onto Hayward and Tomuri (Selection 258).
Plant:
   Age of the plant described.—Approximately 3 years.
   Sex expression.—Female.
   Ploidy.—Diploid.
   Vigor.—Weak-medium.
   Young shoot color.—Green.
   Young shoot texture.—Density of hair medium.
   Cane diameter.—Average approximately 0.3-0.4 cm.
   Cane texture.—Substantially no hair.
   Trunk diameter.—Average approximately 3 cm in 3 years old plant.
   Stem lenticels.—Medium number and size of lenticels, irregularly spaced and sized. Oblong in shape, color grey-green.
Foliage:
   Leaf.—Length — Approximately 2.5-16 cm; Average length — Approximately 12 cm.
   Width.—Approximately 10-14 cm; Average width — Approximately 10 cm.
   Shape of blade.—Obovate.
   Apex.—Emarginated with cuspidate and rounded.
   Base.—Touching each other.
   Attachment.—Petioled.
   Margin.—Very few ciliate.
   Texture of top surface.—Smooth hairless.
   Texture of underside.—Pubescent, slightly velvety.
   Color.—Intense green. Mature foliage under side: light green.
   Petiole.—Length: Average length approximately 8 cm; Average diameter approximately 5 mm.
Flower:
   Flowers per inflorescence.—Normally one.
   Bud color.—Green.
   Bud break.—Mid-March.
   First flower.—End-April.
   Diameter.—Average approximately 4-4.2 cm.
   Petal quantity.—Average 5-7 per flower. Apex curvature: weakly expressed (rounded apex).
   Petals overlapping.—Petals touching, sometimes slightly overlapping.
   Color.—White.
   Filament color.—White.
   Another color.—Yellow-green.
   Attitude of styles.—Semi-erect.
   Style color.—White.
   Hair on ovary.—Pubescent, slightly velvety.
   Color of ovary.—White-yellow.
   Number of sepals.—Average 6.
   Color of sepals.—Green.
   Sepal width.—Average approximately 0.5 cm.
   Sepal length.—Average approximately 0.7 cm.
   Sepal texture.—Smooth.
   Peduncle.—Length — Average approximately 4 cm; Color — Yellow-green; Texture — Smooth (slightly pubescent).
Fruit:
   Color outer pericarp.—Yellow.
   Color inner pericarp.—Red.
   Core color.—White.
   Brix at consumption.—Average approximately 19.0.
   Brix at harvest.—Average approximately 8.5.
   Brix at the beginning of September.—Average approximately 6.
   Average weight.—Approximately 100 grams.
   Minimum weight.—Approximately 70 grams.
   Maximum weight.—Approximately 120 grams.
   Length.—Average approximately 6.5 cm.
   Width.—Average approximately 5.5 cm.
   Core diameter (maximum).—Approximately 3 cm.
   Core diameter (minimum).—Approximately 1 cm.
   Fruit peduncle length.—Average approximately 4.2 cm.
   Fruit peduncle width.—Average approximately 0.3 cm.
   General shape.—Oblong.
   Shoulder shape.—Obovate (slightly pear shaped with the top slightly less wide than the bottom).
   Calyx ring.—Present.
   Calyx ring expression.—Weakly-expressed.
   Skin color at harvest.—Green-yellow-brown. With more sun, the color is more brown. With less sun, the color is more green-yellow.
   Hair on fruit skin.—Pubescent, small and short hair.
   Hair adherence to skin.—Weak.
   Skin adherence to flesh at maturity.—Moderate.
   Fruit core shape.—Transversely oblate.
   Core-woody spike.—Absent.
   Lenticels on fruit.—Minute, moderate in quantity, colored yellow-green.
   Mature seed color.—Black.
   Dried seed color.—Brown.
   Usual harvest time.—2^{nd} decade (10^{th}-20^{th}) of October.
   Overall cropping quantity.—30 tons per hectare.
Other characteristics:
   Storage life.—Storage life is a minimum of about 5 months at 1 degree C.
   Disease/pest resistance.—Neither resistance, nor susceptibility to pathogens and pests common to Actinidia chinensis Planchon have been observed.
   Temperature tolerance.—Tolerates low temperatures to approximately −5 degrees C. (in winter, not in spring) and higher without negative effects; tolerates high temperatures to approximately 40 degrees C. without negative effects.

What is claimed is:

1. A new and distinct variety of kiwi (Actinidia chinensis Planchon) named ‘RED GOAL’, as illustrated and described herein.
